(19) United States
(12) Patent Application Publication
(10) Pub. No.: US 2014/0309167 A1
(43) Pub. Date: Oct. 16, 2014

(54) COMBINATION OF ACTIVE INGREDIENTS FOR THE TREATMENT OF ACUTE KIDNEY INJURY

(71) Applicants: Fabio Sallustio, Bari (BA) (IT); Vincenzo Costantino, Bari (BA) (IT); Francesco Paolo Schena, Bari (BA) (IT)

(72) Inventors: Fabio Sallustio, Bari (BA) (IT); Vincenzo Costantino, Bari (BA) (IT); Francesco Paolo Schena, Bari (BA) (IT)

(73) Assignee: UNIVERSITA ; DEGLI STUDI BARI, Bari (IT)

(21) Appl. No.: 14/353,236

(22) PCT Filed: Nov. 9, 2011
(86) PCT No.: PCT/IB2011/002637

§ 371 Date: Jun. 17, 2014

(51) Int. Cl. A61K 38/18; A61K 38/17

(52) U.S. Cl. CPC A61K 38/1841; A61K 38/1825; A61K 38/1709
USPC 514/8.9

(57) ABSTRACT

The present invention relates to a new combination for the treatment of renal damage, in particular of acute renal failure and acute kidney injury, the pharmaceutical compositions and the kits containing it and their use in therapy.

### SUMMARY OF THE INVENTION

The present invention relates to a new combination of active ingredients for the treatment of acute renal failure and acute kidney injury, the pharmaceutical compositions and the kits containing it and their use in therapy.

### TECHNICAL BACKGROUND

Acute Renal Failure (ARF) is emerging as a major world health problem; it represents the most frequent complication in patients admitted to intensive care units and also has a high mortality rate. Acute renal failure is present in 35-65% of intensive care patients and in 5-20% of hospitalised patients in general. Mortality rate increases considerably in the presence of acute kidney injury; in fact, the majority of studies show a threefold increase in the risk of death for patients suffering from ARF with respect to patients who are not affected by ARF.

Depending on the severity and the duration of the attack, tubular damage can regress, although a critical number of cells must survive to restore structural integrity.

The repair mechanisms reflect different levels of damage, managed differently by the kidney. A first degree of tissue damage at DNA level is rectified by DNA-polymerase, associated with other mechanisms for the repair of misalignment and/or the substitution of nucleotides. If the DNA cannot be repaired, the altered cells are eliminated by apoptosis, a fundamental mechanism also in the elimination of intrinsic cells and of infiltrating leukocytes following ischaemia, obstruction, immunological, toxic or chemical damage. A second level of repair is linked to the proliferation of surviving cells. Therefore, apoptosis and acute tubular necrosis play a key role in the processes the lead to acute renal failure following ischaemia or nephrotoxic attack, The response of the renal tubular cells that survive the damage is thought to be fundamental in order to restore renal function.

Currently, the only treatment in use is Continuous Renal Replacement Therapy (CRRT). This generic term refers to any extracorporeal blood purification therapy aimed at substituting the reduced renal function for an indefinite period of time. This treatment given for 24 hours a day improves renal recovery following ARF.

Although different pharmacological therapies have been used to attempt to accelerate recovery and improve survival rates, these have proved effective in experimental models, but have failed to show any benefit in clinical trials and to date there are no existing pharmaceutical agents capable of stimulating or truly improving renal regeneration.

### OBJECTS OF THE INVENTION

An object of the present invention is to provide a new combination of substances capable of contributing to the repair and regeneration of renal tubular cells and its use in the treatment and/or in the prevention of renal damage, in particular in the treatment and/or in the prevention of acute renal failure or acute kidney injury.

Another object of the present invention is to provide pharmaceutical compositions and kits comprising said combination with pharmaceutically acceptable excipients and carriers.

Yet another object of the invention is to provide a method for the treatment and/or prevention of renal damage, in particular of acute renal failure and acute kidney injury, which comprises administration of the combination and of the composition of the invention.

### DETAILED DESCRIPTION OF THE INVENTION

It has now unexpectedly been found that the new combination of active ingredients of the invention has a repairing action on damaged renal tissue. In particular, it has been found that the substances of the combination of the invention act in a complementary manner, and have a surprising regenerative effect on renal tubular damage.

Therefore, the present invention relates to a new combination of active ingredients composed of
fibroblast growth factor-2 (FGF-2);
inhibin beta (INHB-A);
mRNA which codes for cyclin D1 (CCND1) and/or the protein CCND1; and
mRNA which codes for decorin (DCN) and/or the protein DCN.

“Combination” in intended here to indicate that the active ingredients listed above are administered to the subject requiring pharmacological treatment more or less simultaneously or, in any case, when this is not possible, in a very short period of time, so that all the active ingredients are present in the organism simultaneously and can thus exert their complementary action.

The active ingredients constituting the combination of the invention are known per se in the art and are commercially available.

In particular, FGF-2 is an important growth factor involved in regeneration processes.

INHB-A belongs to the TGF (Transforming Growth Factor) family and performs a prominent role in tissue development processes.

CCND1 is a protein involved mainly in cell proliferation mechanisms and is responsible for the migration of cells from phase G1 to phase S of the cell cycle.

DCN is a protein known for its repairing role following pathological processes and for its angiogenic action.

According to an embodiment of the invention, the combination is constituted by FGF-2, INHB-A, CCND1 protein and DCN protein.

According to another embodiment of the invention, the combination is constituted by FGF-2, INHB-A, mRNA which codes for CCND1 and mRNA which codes for DCN.

The combination of the invention is useful for the treatment of renal damage, in particular acute renal failure and acute kidney injury.

Therefore, according to another of its aspects, the invention relates to the combination of the invention for its use in the treatment and/or in the prevention of renal damage, in particular in the treatment and/or in the prevention of acute renal failure and acute kidney injury.

The composition is also useful in the prevention of renal damage, for example by administration of the composition for preventive purposes immediately prior to a surgical operation with risk of renal damage, advantageously of acute kidney injury. As stated, it has been seen that the active ingredients of the combination perform an efficacious and complementary action. In fact, it has been noted that if one of the active ingredients of the combination is missing, no significant regenerating effect is obtained.

To be administered, the combination is advantageously formulated in a pharmaceutical composition, prepared according to techniques known to those skilled in the art.

According to another of its aspects, the invention relates to a pharmaceutical composition, comprising, as the active ingredient, the combination of the invention, with one or more pharmaceutically acceptable excipients and/or carriers. The preparation of pharmaceutical compositions comprising the combination of active ingredients of the present invention is within the capabilities of those skilled in the art.

In the composition of the invention, the quantities of active substances can vary, naturally according to the weight of the subject to be treated and the severity of the pathology.

By way of example, they can be as follows:
FGF2: from 30 μg/Kg to 36 mg/Kg of body weight, for example approximately from 100 μg/Kg to 10 mg/kg, for example approximately from 500 μg/Kg to 1 mg/kg;
Inhibin A: from 0.5 μg/kg to 3 mg/Kg of body weight, for example approximately from 10 μg/Kg to 1 mg/kg, of body weight, for example approximately from 100 μg/Kg to 0.5 mg/kg;
CCND1 and DCN in protein form: from 20 μg/Kg to 30 mg/Kg of body weight, for example approximately from 100 μg/Kg to 10 mg/kg, of body weight, for example approximately from 500 μg/Kg to 1 mg/kg.

When CCND1 and DCN are administered in the form of mRNA, the quantities of each substance can vary, for example between 200 ng-10 μg/kg of body weight, for example approximately 500 ng-1 μg/kg.

The frequency of the administrations can vary according to the severity of the damage and the response of the treated subject, and once daily administration, administration every two days or even a single weekly administration can be given. However, doses and dosages can be adjusted and optimized by those skilled in the art on the basis of results and improvements occurring during the therapy.

The protein part of the combination and/or the composition of the invention can be administered, for example, using an infusion pump through standard diagnostic catheters or by intravenous or intra-arterial administration.

When wishing to use CCND1 and/or DCN in the form of mRNA, these can be administered in the form of liposome complexes or in SNALP delivery systems.

According to the nature of the substances or also only if desirable, the combination can be formulated in a single composition or in a plurality of pharmaceutical compositions, to be administered simultaneously or in any case within a short period of time.

The present invention also relates to a kit comprising the combination of the invention in which the active substances are contained in two or more different pharmaceutical compositions.

According to an embodiment, the kit of the invention comprises the protein substances of the combination in pharmaceutical form and the mRNA or mRNAs in a different pharmaceutical form, these compositions being, for example, of the type described above.

Advantageously, the kit also comprises an information leaflet.

The subject to be treated with the combination, the composition or the kit of the invention is an animal, generally a mammal, and more advantageously, but not only, a human being. In fact, the combination and the composition of the invention can also be administered to animals requiring a similar treatment.

According to another of its aspects, the invention relates to the combination, the composition or the kit of the invention for use in the treatment of acute renal failure and acute kidney injury.

According to another of its aspects, the invention relates to a method for the treatment and/or prevention of acute renal failure and acute kidney injury which comprises the administration, to a subject requiring it, of an effective quantity of the combination or of the composition of the invention.

The efficacy of the combination was tested in experiments on primary tubular cells, deriving from human renal tissue, (RPTEC), and on immortalized tubular cells such as Human Kidney 2 (HK2).

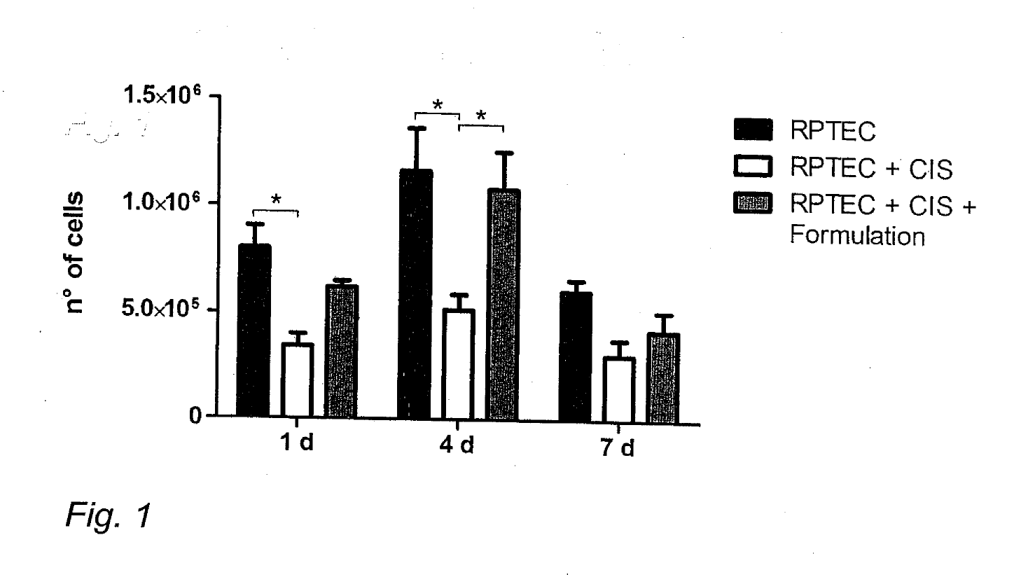

In particular, RPTEC or HK2 were subjected to damage induced by cisplatin in a culture model in vitro of said cells together with the combination for 1, 4 and 7 days from induction of the damage. In the model in vitro, RPTEC (or HK2) were grown on the bottom of the wells of a 9.5 cm² plate; these cells were induced to apoptosis through the cytotoxic action of cisplatin and subsequently placed in a culture with the combination. RPTEC and HK2 not subjected to the action of cisplatin were used as negative control and those subjected only to the action of cisplatin were used as positive control. As shown in FIG. 1, exposure of RPTEC or HK2 to cisplatin greatly reduced cell number and vitality, but the culture with the combination provided a protective effect, promoting proliferation and preventing apoptosis of the tubular cells. For this purpose, cell counts were carried out at different intervals of time from induction of the damage and the expression in parallel of typical proliferation markers (Ki67) and apoptotic markers (Caspase 3) were assessed through Immunofluorescence assays and Confocal Microscopy.

The same type of experiment was carried out with FGF2 in place of the combination of the invention. The results showed cell recovery with respect to the controls treated only with cisplatin, although the increase in the number of cells was in any case insignificant.

The same type of experiment was also carried out with the combination without INHB-A. The results show extremely low cell recovery.

1. A combination of active ingredients composed of
fibroblast growth factor-2 (FGF-2);
inhibin beta (INHB-A);
mRNA which codes for cyclin D1 (CCND1) and/or the protein CCND1; and
mRNA which codes for decorin (DNC) and/or the protein DCN).

2. The combination of claim 1, consisting of FGF-2, INHB-A, CCND1 protein and DCN protein.

3. The combination of claim 1, consisting of FGF-2, INHB-A, mRNA which codes for CCND1 and mRNA which codes for DCN.

4. The combination of claim 1, for its use in the treatment and/or in the prevention of renal damage.

5. The combination for its use according to claim 4, in the treatment and/or in the prevention of acute renal failure or of acute kidney injury.

6. A pharmaceutical composition, comprising, as active ingredient, the combination of claim 1, with one or more pharmaceutically acceptable excipients and/or carriers.

7. The composition according to claim 5, for its use in the treatment and/or in the prevention of renal damage.

8. The composition for its use according to claim 7, in the treatment and/or in the prevention of acute renal failure or of acute kidney injury.

9. A kit comprising the combination of claim 1, in which the active substances are contained in two or more different pharmaceutical compositions.

10. The kit according to claim 8 comprising the protein substances of the combination in pharmaceutical form and the mRNA or mRNAs in a different pharmaceutical form.
